(19) United States
(12) Patent Application Publication
(10) Pub. No.: US 2010/0190657 A1
(43) Pub. Date: Jul. 29, 2010

(54) Oligonucleotide-tagged semiconductor nanocrystals for microarray and fluorescence in situ hybridization

(75) Inventors: Michael BITTNER, Rockville, MD (US); Edith Y. Wong, Danville, CA (US); Marcel P. Bruchez, JR., Pittsburgh, PA (US)

Correspondence Address: LIFE TECHNOLOGIES CORPORATION;C/O INTELLEVATE, P.O. BOX 52050, MINNEAPOLIS, MN, 55402, US

(73) Assignee: LIFE TECHNOLOGIES CORPORATION, Carlsbad, CA (US)

(21) Appl. No.: 12/604,296

(22) Filed: Oct. 22, 2009

Related U.S. Application Data
(63) Continuation of application No. 09/766,273, filed on Jan. 18, 2001, now abandoned.
(60) Provisional application No. 60/176,793, filed on Jan. 18, 2000.

(51) Int. Cl. C40B 30/04; C12Q 1/68

(52) U.S. Cl. 506/9; 435/6

(57) ABSTRACT

Methods for assaying a sample for a probe polynucleotide are provided. The methods comprise forming a complex between a target on a substrate, the probe polynucleotide that binds to the target, and a conjugate comprising a semiconductor nanocrystal that binds to the probe polynucleotide by way of a tag sequence on the probe polynucleotide. The complex is formed when the probe polynucleotide is present in the sample. The methods are useful in any technique in which the detection of a target that can bind to a probe polynucleotide is desired, for example in fluorescence in situ hybridization. The methods are particularly useful in multiplex settings such as hybridization to microarrays where a plurality of targets are present. Assay complexes produced by such methods and kits useful for performing such methods are also provided.

### TECHNICAL FIELD

This invention relates to methods, articles and compositions for the analysis of polynucleotides in a sample.

### BACKGROUND OF THE INVENTION

Sequencing projects directed towards obtaining the complete genomic sequences of various organisms have led to a huge proliferation of available sequence data. This data has created an even greater need for efficient methods of performing experiments designed to discover and characterize the functions of the numerous new genes being identified, and the effects various perturbations have on their expression. Although methods currently existing for performing such experiments, they are limited in a number of ways.

Typical polynucleotide analysis methods involve the use of traditional labels, frequently fluorescent labels to detect various binding events. Traditional fluorescent labels are small organic molecules which suffer from a number of limitations, including a tendency to bind to surfaces non-specifically and produce increased background signals, limited spectral characteristics, and relatively short useful lifetimes due to photobleaching. Background signals limit both the sensitivity and the accuracy of assays. Limited spectral characteristics allow fewer different labels to be used without interference, and thus fewer assays to be performed simultaneously on the same sample. This limits the amount of multiplexing that can be built into a given assay. Furthermore, techniques which involve enzymatic incorporation of labeled nucleotides into polynucleotides suffer from variations in enzyme processivity and in the amount of label incorporated.

Many of these techniques require a unique conjugated polynucleotide to be prepared for each experiment. These require either a costly and inefficient chemical coupling of the label to a polynucleotide, or an enzymatic incorporation which suffers from assay to assay and probe to probe variability, and thus limits the accuracy and reproducibility of such assays.

There is a need in the art for methods of analyzing samples for polynucleotides, and for devices, compositions and articles of manufacture useful in such methods.

### SUMMARY OF THE INVENTION

Methods for assaying a sample for a probe polynucleotide are provided. The methods involve contacting a target molecule, typically a target polynucleotide, with a sample suspected of containing a probe polynucleotide. The target is attached to a substrate. The probe polynucleotide comprises a tag sequence which does not bind to the target. The tag sequence allows for the probe polynucleotide to be used as a bridge between the target and a tag-binding conjugate which comprises a semiconductor nanocrystal. The tag-binding conjugate also comprises a tag-binding sequence that can hybridize to the tag sequence under assay conditions. The binding of these reagents together forms a probe assay complex which is useful for determining the presence and/or amount of the probe polynucleotide in the sample. Kits which can be used for performing the methods of the invention are also provided.

The methods are particularly useful in multiplex settings where a plurality of different targets are used to assay for a plurality of different probe polynucleotides. The large number of distinguishable semiconductor nanocrystal labels allows for the simultaneous analysis of multiple probe polynucleotides.

The generic tag-binding conjugates are not probe-specific, and suitable preparation schemes can incorporate the same tag sequences into different polynucleotides used in the same or different assay. This alleviates the need to label each of a plurality of probe polynucleotides individually for each assay.

### BRIEF DESCRIPTION OF THE DRAWINGS

FIG. 1 is a depiction of the preparation of cDNA from RNA derived from a test sample and a reference sample using first and second DNA primers comprising first and second respective tag sequences.

FIG. 2 is a depiction of a first hybridization step in which first and second cDNAs comprising first and second respective tag sequences are incubated with a substrate to which has been attached target DNA sequences.

FIG. 3 is a depiction of a second hybridization step in which the substrate is further incubated with first and second semiconductor nanocrystal (“SCNC”)-tag-binding conjugates which are complementary to the first and second tag sequences, respectively.

FIG. 4 is a pictorial representation of the layout of a microarray slide in which four synthetic complementary gene fragments 70 nucleotides in length (70-mer) are spotted at ten different locations on a solid support.

FIG. 5A is a pictorial illustration showing one possible assay scheme using the microarray depicted in FIG. 4. FIG. 5B is a photographic image of the microarray of FIG. 4 after hybridization exposed to light at an excitation wavelength of 488 nm in which hybridization of a green SCNC-DNA conjugate to Gene A is detected at an emission wavelength of 522 nm. FIG. 5C is a photographic image of the microarray of FIG. 4 after hybridization exposed to light at an excitation wavelength of 488 nm in which hybridization of a red SCNC-DNA probe to Genes A and B is detected at an emission wavelength of 630 nm.

FIG. 6A is a pictorial illustration similar to FIG. 5A, but further depicting a biotinylated DNA probe bound to a 592 nm yellow SCNC-streptavidin conjugate. FIG. 6B is a pictorial illustration of the layout of synthetic 70-mer complementary gene fragments on a microarray slide. FIG. 6C is a photographic image of a microarray exposed to light at an excitation wavelength of 488 nm in which hybridization of a green SCNC-DNA conjugate to Genes A and B is detected at an emission wavelength of 522 nm. FIG. 6D is a photographic image of a microarray exposed to light at an excitation wavelength of 488 nm in which hybridization of a red SCNC-DNA conjugate to Genes A and B is detected at an emission wavelength of 630 nm. FIG. 6E is a photographic image of a microarray exposed to light at an excitation wavelength of 488 nm in which hybridization of a yellow SCNC-streptavidin conjugate bound to a biotinylated DNA probe complementary to Gene A is detected at an emission wavelength of 592 nm.

FIG. 7 depicts a slide having two microarrays available for hybridization.

FIG. 8. Aliquots of total RNA isolated from the melanoma cell line UACC903 were labeled with Cy3 and Cy5 using the standard oligo dT primer, or an oligo dT primer with a 5′ extension specifying the phage 186 cos site. The labeled cDNA was hybridized to a cDNA chip composed of 6240 named human genes. The mean intensity of the signals for each detected gene is plotted. The dotted lines denote the bounds of the 99.99% confidence interval for the ratios of the observed values (0.64 to 1.59).

### DETAILED DESCRIPTION OF THE INVENTION

The invention provides methods in which a target attached to a substrate is contacted with a sample suspected of comprising a probe polynucleotide under conditions in which the probe polynucleotide can bind to the target. The target is typically a polynucleotide, but may be another species, for example a protein, an antibody, or a DNA-binding protein, that can bind to a polynucleotide, particularly when the probe polynucleotide is an aptamer. The probe polynucleotide comprises a tag sequence which does not bind to the target. The probe polynucleotide does not require tedious coupling reactions to introduce a label, but can be automatically synthesized to incorporate the novel tag sequence cheaply and efficiently. The sample is also contacted with a tag-binding conjugate, which comprises a semiconductor nanocrystal conjugated to a tag-binding sequence that can bind to the tag on the probe polynucleotide; this step may be performed prior to or concurrently with the step of contacting the substrate with the sample, but is typically done after. Wash steps are preferably performed after each contacting step to minimize background, but alternatives such as dilution may be used. In some instances, no washing or dilution may be needed if an acceptable signal can be achieved. A light source that can excite the semiconductor nanocrystal in the tag-binding is applied to the substrate, and the substrate is assayed to determine if the semiconductor nanocrystal is associated with the substrate, thereby indicating whether the probe polynucleotide was present in the sample. The amount of probe polynucleotide in the sample can also be determined.

Because of the large number of different semiconductor nanocrystals and mixtures thereof which can be distinguished, large numbers of different probe polynucleotides and targets can be simultaneously interrogated. Furthermore, because the semiconductor nanocrystals can be incorporated into generic tag-binding conjugates, those tags may be used in different assays for different probe polynucleotides, thus allowing for standardization in labeling and simplifying the preparation of a new probe polynucleotide for a new experiment. Multiplex methods are also provided employing 2, 3, 4, 5, 10 15, 20, 25, 50, 100, 200, 500, 1000 or more different probe polynucleotides, which are limited only by the number of different semiconductor nanocrystal labels that can be distinguished, can be used simultaneously with corresponding different targets and different tag-binding conjugates. As these labels can be distinguished in 1 nm intervals throughout the visible spectrum, combinations and different ratios within those combinations allow for a large number of distinguishable tag-binding conjugates to be prepared.

Also provided are probe polynucleotide assay complexes produced by such methods, and kits comprising components useful for such methods.

The inventions described herein can be used for any assay in which a sample is interrogated regarding a probe polynucleotide. Typical assays might involve determining the presence of the probe polynucleotide in the sample, the relative amount of the probe polynucleotide, or may be quantitative or semi-quantitative regarding the amount of probe polynucleotide in the sample to study alterations of gene expression in response to a stimulus, differences between different samples. The inventions can be used in Northern blots, Southern blots, fluorescence in situ hybridizations, and in microarrays. DNA screening is also used in a variety of other areas, including carrier screening, prenatal diagnostic testing, newborn screening, presymptomatic testing for adult-onset diseases and disorders such as cancer, Huntington's disease and Alzheimer's disease, paternity tests and in forensic identifications.

Before the present invention is described in detail, it is to be understood that this invention is not limited to the particular methodology, devices, solutions or apparatuses described, as such methods, devices, solutions or apparatuses can, of course, vary. It is also to be understood that the terminology used herein is for the purpose of describing particular embodiments only, and is not intended to limit the scope of the present invention.

Use of the singular forms “a,” “an,” and “the” include plural references unless the context clearly dictates otherwise. Thus, for example, reference to “a target” includes a plurality of targets, reference to “a substrate” includes a plurality of such substrates, reference to “a probe” includes a plurality of probes, and the like.

Terms such as “connected,” “attached,” “linked,” and “conjugated” are used interchangeably herein and encompass direct as well as indirect connection, attachment, linkage or conjugation unless the context clearly dictates otherwise. Where a range of values is recited, it is to be understood that each intervening integer value, and each fraction thereof, between the recited upper and lower limits of that range is also specifically disclosed. The upper and lower limits of any range can independently be included in or excluded from the range, and each range where either, neither or both limits are included is also encompassed within the invention. Where a value being discussed has inherent limits, for example where a component can be present at a concentration of from 0 to 100%, or where the pH of an aqueous solution can range from 1 to 14, those inherent limits are specifically disclosed. Where a value is explicitly recited, it is to be understood that values which are about the same quantity or amount as the recited value are also within the scope of the invention. Where a combination is disclosed, each subcombination of the elements of that combination is also specifically disclosed and is within the scope of the invention. For any element of an invention for which a plurality of options are disclosed, examples of that invention in which each of those options is individually excluded along with all possible combinations of excluded options are hereby disclosed.

Unless defined otherwise or the context clearly dictates otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

All publications mentioned herein are hereby incorporated by reference for the purpose of disclosing and describing the particular materials and methodologies for which the reference was cited. The publications discussed herein are provided solely for their disclosure prior to the filing date of the present application. Nothing herein is to be construed as an admission that the invention is not entitled to antedate such disclosure by virtue of prior invention.

### DEFINITIONS

In describing the present invention, the following terms will be employed, and are intended to be defined as indicated below.

The terms “semiconductor nanocrystal,” “SCNC,” “quantum dot” and “Qdot™ nanocrystal” are used interchangeably herein and refer to an inorganic crystallite of about 1 nm or more and about 1000 nm or less in diameter or any integer or fraction of an integer therebetween, preferably at least about 2 nm and about 50 nm or less in diameter or any integer or fraction of an integer therebetween, more preferably at least about 2 nm and about 20 nm or less in diameter (for example about 2, 3, 4, 5, 6, 7, 8, 9, 10, 11, 12, 13, 14, 15, 16, 17, 18, 19, or nm). SCNCs are characterized by their uniform nanometer size. An SCNC is capable of emitting electromagnetic radiation upon excitation (i.e., the SCNC is luminescent) and includes a “core” of one or more first semiconductor materials, and may be surrounded by a “shell” of a second semiconductor material. An SCNC core surrounded by a semiconductor shell is referred to as a “core/shell” SCNC. The surrounding “shell” material will preferably have a bandgap energy that is larger than the bandgap energy of the core material and may be chosen to have an atomic spacing close to that of the “core” substrate. The core and/or the shell can be a semiconductor material including, but not limited to, those of the group II-VI (ZnS, ZnSe, ZnTe, CdS, CdSe, CdTe, HgS, HgSe, HgTe, MgS, MgSe, MgTe, CaS, CaSe, CaTe, SrS, SrSe, SrTe, BaS, BaSe, BaTe, and the like) and III-v (GaN, GaP, GaAs, GaSb, InN, InP, InAs, InSb, and the like) and IV (Ge, Si, and the like) materials, and an alloy or a mixture thereof.

An SCNC is optionally surrounded by a “coat” of an organic capping agent. The organic capping agent may be any number of materials, but has an affinity for the SCNC surface. In general, the capping agent can be an isolated organic molecule, a polymer (or a monomer for a polymerization reaction), an inorganic complex, or an extended crystalline structure. The coat can be used to convey solubility, e.g., the ability to disperse a coated SCNC homogeneously into a chosen solvent, functionality, binding properties, or the like. In addition, the coat can be used to tailor the optical properties of the SCNC.

Thus, the terms “semiconductor nanocrystal,” “SCNC,” “quantum dot” and “Qdot™ nanocrystal” as used herein include a coated SCNC core, as well as a core/shell SCNC.

“Monodisperse particles” include a population of particles wherein at least about 60% of the particles in the population, more preferably about 75 to about 90, or any integer therebetween, percent of the particles in the population fall within a specified particle size range. A population of monodisperse particles deviates less than 10% rms (root-mean-square) in diameter, and preferably deviates less than 5% rms.

The phrase “one or more sizes of SCNCs” is used synonymously with the phrase “one or more particle size distributions of SCNCs.” One of ordinary skill in the art will realize that particular sizes of SCNCs are actually obtained as particle size distributions.

The terms “polynucleotide,” “oligonucleotide,” “nucleic acid” and “nucleic acid molecule” are used herein to include a polymeric form of nucleotides of any length, and may comprise ribonucleotides, deoxyribonucleotides, analogs thereof, or mixtures thereof. This term refers only to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), including tRNA, rRNA, hRNA, and mRNA, whether spliced or unspliced, any other type of polynucleotide which is an N- or C-glycoside of a purine or pyrimidine base, and other polymers containing nonnucleotidic backbones, for example, polyamide (e.g., peptide nucleic acids (PNAs)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms are used interchangeably herein. These terms refer only to the primary structure of the molecule. Thus, these terms include, for example, 3′-deoxy-2′,5′-DNA, oligodeoxyribonucleotide N3′P5′ phosphoramidates, 2′-O-alkyl-substituted RNA, double- and single-stranded DNA, as well as double- and single-stranded RNA, and hybrids thereof including for example hybrids between DNA and RNA or between PNAs and DNA or RNA, and also include known types of modifications, for example, labels, alkylation, “caps,” substitution of one or more of the nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalkylphosphoramidates, aminoalkylphosphotriesters), those containing pendant moieties, such as, for example, proteins (including enzymes (e.g. nucleases), toxins, antibodies, signal peptides, poly-L-lysine, etc.), those with intercalators (e.g., acridine, psoralen, etc.), those containing chelates (of, e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide.

It will be appreciated that, as used herein, the terms “nucleoside” and “nucleotide” will include those moieties which contain not only the known purine and pyrimidine bases, but also other heterocyclic bases which have been modified. Such modifications include methylated purines or pyrimidines, acylated purines or pyrimidines, or other heterocycles. Modified nucleosides or nucleotides can also include modifications on the sugar moiety, e.g., wherein one or more of the hydroxyl groups are replaced with halogen, aliphatic groups, or are functionalized as ethers, amines, or the like. The term “nucleotidic unit” is intended to encompass nucleosides and nucleotides.

Furthermore, modifications to nucleotidic units include rearranging, appending, substituting for or otherwise altering functional groups on the purine or pyrimidine base which form hydrogen bonds to a respective complementary pyrimidine or purine. The resultant modified nucleotidic unit optionally may form a base pair with other such modified nucleotidic units but not with A, T, C, G or U. Abasic sites may be incorporated which do not prevent the function of the polynucleotide. Some or all of the residues in the polynucleotide can optionally be modified in one or more ways.

Standard A-T and G-C base pairs form under conditions which allow the formation of hydrogen bonds between the N3-H and C4-oxy of thymidine and the N1 and C6-NH2, respectively, of adenosine and between the C2-oxy, N3 and C4-NH2, of cytidine and the C2-NH2, N′—H and C6-oxy, respectively, of guanosine. Thus, for example, guanosine (2-amino-6-oxy-9-β-D-ribofuranosyl-purine) may be modified to form isoguanosine (2-oxy-6-amino-9-β-D-ribofuranosyl-purine). Such modification results in a nucleoside base which will no longer effectively form a standard base pair with cytosine. However, modification of cytosine (1-β-D-ribofuranosyl-2-oxy-4-amino-pyrimidine) to form isocytosine (1-β-D-ribofuranosyl-2-amino-4-oxy-pyrimidine) results in a modified nucleotide which will not effectively base pair with guanosine but will form a base pair with isoguanosine. Isocytosine is available from Sigma Chemical Co. (St. Louis, Mo.); isocytidine may be prepared by the method described by Switzer et al. (1993) Biochemistry 32:10489-10496 and references cited therein; 2′-deoxy-5-methyl-isocytidine may be prepared by the method of Tor et al. (1993) J. Am. Chem. Soc. 115:4461-4467 and references cited therein; and isoguanine nucleotides may be prepared using the method described by Switzer et al. (1993), supra, and Mantsch et al. (1993) Biochem. 14:5593-5601, or by the method described in U.S. Pat. No. 5,780,610 to Collins et al. Other nonnatural base pairs may be synthesized by the method described in Piccirilli et al. (1990) Nature 343:33-37 for the synthesis of 2,6-diaminopyrimidine and its complement (1-methylpyrazolo-[4,3]pyrimidine-5,7-(4H,6H)-dione. Other such modified nucleotidic units which form unique base pairs are known, such as those described in Leach et al. (1992) J. Am. Chem. Soc. 114:3675-3683 and Switzer et al., supra.

“Nucleic acid probe” and “probe” are used interchangeably and refer to a structure comprising a polynucleotide, as defined above, that contains a nucleic acid sequence that can bind to a corresponding target. The polynucleotide regions of probes may be composed of DNA, and/or RNA, and/or synthetic nucleotide analogs.

“Complementary” or “substantially complementary” refers to the hybridization or base pairing between nucleotides or nucleic acids, such as, for instance, between the two strands of a double stranded DNA molecule or between a polynucleotide primer and a primer binding site on a single stranded nucleic acid to be sequenced or amplified: Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%.

Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984).

Stringent hybridization conditions will typically include salt concentrations of less than about 1M, more usually less than about 500 mM and preferably less than about 200 mM. Hybridization temperatures can be as low as 5° C., but are typically greater than 22° C., more typically greater than about 30° C., and preferably in excess of about 37° C. Longer fragments may require higher hybridization temperatures for specific hybridization. Other factors may affect the stringency of hybridization, including base composition and length of the complementary strands, presence of organic solvents and extent of base mismatching, and the combination of parameters used is more important than the absolute measure of any one alone.

The terms “aptamer” (or “nucleic acid antibody”) is used herein to refer to a single- or double-stranded polynucleotide that recognizes and binds to a desired target molecule by virtue of its shape. See, e.g., PCT Publication Nos. WO 92/14843, WO 91/19813, and WO 92/05285.

“Polypeptide” and “protein” are used interchangeably herein and include a molecular chain of amino acids linked through peptide bonds. The terms do not refer to a specific length of the product. Thus, “peptides,” “oligopeptides,” and “proteins” are included within the definition of polypeptide. The terms include polypeptides contain [post-translational] modifications of the polypeptide, for example, glycosylations, acetylations, phosphorylations, and sulphations. In addition, protein fragments, analogs (including amino acids not encoded by the genetic code, e.g. homocysteine, ornithine, D-amino acids, and creatine), natural or artificial mutants or variants or combinations thereof, fusion proteins, derivatized residues (e.g. alkylation of amine groups, acetylations or esterifications of carboxyl groups) and the like are included within the meaning of polypeptide.

The terms “substrate” and “support” are used interchangeably and refer to a material having a rigid or semi-rigid surface.

As used herein, the term “binding pair” refers to first and second molecules that bind specifically to each other with greater affinity than to other components in the sample. The binding between the members of the binding pair is typically noncovalent. Exemplary binding pairs include immunological binding pairs (e.g. any haptenic or antigenic compound in combination with a corresponding antibody or binding portion or fragment thereof, for example digoxigenin and anti-digoxigenin, fluorescein and anti-fluorescein, dinitrophenol and anti-dinitrophenol, bromodeoxyuridine and anti-bromodeoxyuridine, mouse immunoglobulin and goat anti-mouse immunoglobulin) and nonimmunological binding pairs (e.g., biotin-avidin, biotin-streptavidin, hormone [e.g., thyroxine and cortisol]-hormone binding protein, receptor-receptor agonist or antagonist (e.g., acetylcholine receptor-acetylcholine or an analog thereof) IgG-protein A, lectin-carbohydrate, enzyme-enzyme cofactor, enzyme-enzyme-inhibitor, and complementary polynucleotide pairs capable of forming nucleic acid duplexes) and the like. One or both member of the binding pair can be conjugated to additional molecules.

An “SCNC conjugate” is an SCNC linked to an oligonucleotide, as defined above.

An SCNC is “linked” or “conjugated” to, or “associated” with, a tag-binding polynucleotide when the SCNC is chemically coupled to, or associated with the tag-binding polynucleotide. Thus, these terms intend that the SCNC may either be directly linked to the tag-binding polynucleotide or may be linked via a linker moiety, such as via a chemical linker. The terms indicate items that are physically linked by, for example, covalent chemical bonds, physical forces such van der Waals or hydrophobic interactions, encapsulation, embedding, or the like. For example, nanocrystals can be associated with biotin which can bind to the proteins avidin and streptavidin.

The term “antibody” as used herein includes antibodies obtained from both polyclonal and monoclonal preparations, as well as: hybrid (chimeric) antibody molecules (see, for example, Winter et al. (1991) Nature 349:293-299; and U.S. Pat. No. 4,816,567); F(ab′)2 and F(ab) fragments; Fv molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (sFv) (see, for example, Huston et al. (1988) Proc Natl Acad Sci USA 85:5879-5883); dimeric and trimeric antibody fragment constructs; minibodies (see, e.g., Pack et al. (1992) Biochem 31:1579-1584; Cumber et al. (1992) J Immunology 149B:120-126); humanized antibody molecules (see, for example, Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

As used herein, the term “monoclonal antibody” refers to an antibody composition having a homogeneous antibody population. The term is not limited regarding the species or source of the antibody, nor is it intended to be limited by the manner in which it is made. Thus, the term encompasses antibodies obtained from murine hybridomas, as well as human monoclonal antibodies obtained using human hybridomas or from murine hybridomas made from mice expression human immunoglobulin chain genes or portions thereof. See, e.g., Cote, et al. Monoclonal Antibodies and Cancer Therapy, Alan R. Liss, 1985, p. 77.

A “homogeneous assay” is one that is performed without transfer, separation or washing steps. Thus, for example, a homogeneous high-throughput screening (“HTS”) assay involves the initial addition of reagents to a vessel, e.g., a test tube or sample well, followed by the detection of the results from that vessel. A homogeneous HTS assay can be performed anywhere in the vessel, for example in the solution, on the surface of the vessel or on beads or surfaces placed in the vessel. The detection system typically used is a fluorescence, chemiluminescence, or scintillation detection system.

“Multiplexing” herein refers to an assay or other analytical method in which multiple probe polynucleotides can be assayed simultaneously by using more than one SCNC, each of which has at least one different fluorescence characteristic (for example excitation wavelength, emission wavelength, emission intensity, FWHM (full width at half maximum peak height), or fluorescence lifetime). Multiplexing also includes assays or methods in which the combination of more than one SCNC having distinct emission spectra can be used to detect a single probe polynucleotide.

For example, two different preparations of SCNCs may have the same composition but different particle sizes, and thus differ in excitation and/or emission wavelength. Or, two different preparations may have the same particle size or particle size distribution but different composition, and thus also differ in excitation and/or emission wavelength. Different preparations having different compositions of SCNCs can have different fluorescent lifetimes, and thus their emission spectra can be distinguished even when they have the same emission wavelength and intensity, for example by sampling the emission from the encoded substance at different times after excitation. Differences in FWHM can be achieved for example by using SCNCs of different composition, or of the same composition but which are synthesized differently, or by mixing different SCNC “preparations” having overlapping emission peaks together to form a new preparation.

A SCNC having a known emission wavelength and/or intensity may be included with the SCNCs conjugated to the tag-binding polynucleotide defined herein to provide an internal standard for calibrating the wavelength and/or intensity of the other SCNC(s) used in the conjugate.

“Optional” or “optionally” means that the subsequently described event or circumstance may or may not occur, and that the description includes instances where the event or circumstance occurs and instances in which it does not. For example, the phrase “optionally surrounded by a ‘coat’ of an organic capping agent” with reference to an SCNC includes SCNCs having such a coat, and SCNCs lacking such a coat.

### The Substrate

The substrate can comprise a wide range of material, either biological, nonbiological, organic, inorganic, or a combination of any of these. For example, the substrate may be a polymerized Langmuir Blodgett film, functionalized glass, Si, Ge, GaAs, GaP, SiO₂, SiN₄, modified silicon, or any one of a wide variety of gels or polymers such as (poly)tetrafluoroethylene, (poly)vinylidenedifluoride, polystyrene, cross-linked polystyrene, polyacrylic, polylactic acid, polyglycolic acid, poly(lactide coglycolide), polyanhydrides, poly(methyl methacrylate), poly(ethylene-co-vinyl acetate), polysiloxanes, polymeric silica, latexes, dextran polymers, epoxies, polycarbonate, or combinations thereof.

Substrates can be planar crystalline substrates such as silica based substrates (e.g. glass, quartz, or the like), or crystalline substrates used in, e.g., the semiconductor and microprocessor industries, such as silicon, gallium arsenide and the like.

Silica aerogels can also be used as substrates, and can be prepared by methods known in the art. Aerogel substrates may be used as free standing substrates or as a surface coating for another substrate material.

The substrate can take any form and typically is a plate, slide, bead, pellet, disk, particle, strand, precipitate, optionally porous gel, sheets, tube, sphere, container, capillary, pad, slice, film, chip, multiwell plate or dish, optical fiber, etc. Although typically the substrate takes an inanimate form, for some applications such as flow cytometry, the substrate can be any form that is rigid or semi-rigid, for example a cell, tissue, organism or nucleus, and may be optionally fixed.

The substrate may contain raised or depressed regions on which a sample is located. The surface of the substrate can be etched using well known techniques to provide for desired surface features, for example trenches, v-grooves, mesa structures, or the like.

Surfaces on the substrate can be composed of the same material as the substrate or can be made from a different material, and can be coupled to the substrate by chemical or physical means. Such coupled surfaces may be composed of any of a wide variety of materials, for example, polymers, plastics, resins, polysaccharides, silica or silica-based materials, carbon, metals, inorganic glasses, membranes, or any of the above-listed substrate materials. In one embodiment, the surface will be optically transparent and will have surface Si—OH functionalities, such as those found on silica surfaces.

The substrate and/or its optional surface are chosen to provide appropriate optical characteristics for the synthetic and/or detection methods used. The substrate and/or surface can be transparent to allow the exposure of the substrate by light applied from multiple directions. The substrate and/or surface may be provided with reflective “mirror” structures to increase the recovery of light emitted by the semiconductor nanocrystal for maximization of emission collected therefrom.

The substrate and/or its surface is generally resistant to, or is treated to resist, the conditions to which it is to be exposed, and can be optionally treated to remove any resistant material after exposure to such conditions.

Targets can be fabricated on or attached to the substrate by any suitable method, for example the methods described in U.S. Pat. No. 5,143,854, PCT WO 92/10092, or U.S. patent application Ser. No. 07/624,120, filed Dec. 6, 1990 (now abandoned), Fodor et al., Science, 251: 767-777 (1991), PCT Application No. WO 90/15070). Techniques for the synthesis of these arrays using mechanical synthesis strategies are described in, e.g., PCT Publication No. 93/09668 and U.S. Pat. No. 5,384,261.

Still further techniques include bead based techniques such as those described in PCT US/93/04145 and pin based methods such as those described in U.S. Pat. No. 5,288,514.

Additional flow channel or spotting methods applicable to attachment of targets to the substrate are described in U.S. patent application Ser. No. 07/980,523, filed Nov. 20, 1992, and U.S. Pat. No. 5,384,261. Reagents are delivered to the substrate by either (1) flowing within a channel defined on predefined regions or (2) “spotting” on predefined regions. A protective coating such as a hydrophilic or hydrophobic coating (depending upon the nature of the solvent) can be used over portions of the substrate to be protected, sometimes in combination with materials that facilitate wetting by the reactant solution in other regions. In this manner, the flowing solutions are further prevented from passing outside of their designated flow paths.

Typical dispensers include a micropipette optionally robotically controlled, an ink jet printer, a series of tubes, a manifold, an array of pipettes, or the like so that various reagents can be delivered to the reaction regions sequentially or simultaneously.

Beads can be immobilized on a planar surface such that they are regularly spaced in a chosen geometry using any suitable method. For example, beads can be immobilized by micromachining wells in which beads can be entrapped into the surface, or by patterned activation of polymers on the surface using light to cross-link single beads at particular locations. Suitable wells can be created by ablating circles in a layer of parylene deposited on a glass surface using a focused laser. The well dimensions are chosen such that a single bead can be captured in a well and remains trapped when a lateral flow of fluid passes across the surface. For example, 7 micron wells can be used for analysis of beads about 4 microns to about 6 microns in diameter. This can be performed on the end of an optical fiber.

### The Sample

The portion of the sample comprising or suspected of comprising the probe polynucleotide can be any source of biological material which comprises polynucleotides that can be obtained from a living organism directly or indirectly, including cells, tissue or fluid, and the deposits left by that organism, including viruses, mycoplasma, and fossils. Typically, the sample is obtained as or dispersed in a predominantly aqueous medium. Nonlimiting examples of the sample include blood, urine, semen, milk, sputum, mucus, a buccal swab, a vaginal swab, a rectal swab, an aspirate, a needle biopsy, a section of tissue obtained for example by surgery or autopsy, plasma, serum, spinal fluid, lymph fluid, the external secretions of the skin, respiratory, intestinal, and genitourinary tracts, tears, saliva, tumors, organs, samples of in vitro cell culture constituents (including but not limited to conditioned medium resulting from the growth of cells in cell culture medium, putatively virally infected cells, recombinant cells, and cell components), and a recombinant library comprising polynucleotide sequences.

The sample can be a positive control sample which is known to contain the probe polynucleotide. A negative control sample can also be used which, although not expected to contain the target polynucleotide is suspected of containing it, and is tested in order to confirm the lack of contamination by the probe polynucleotide of the reagents used in a given assay, as well as to determine whether a given set of assay conditions produces false positives (a positive signal even in the absence of probe polynucleotide in the sample).

The sample can be diluted, dissolved, suspended, extracted or otherwise treated to solubilize any probe polynucleotide present or to render it accessible to reagents which are used in an amplification scheme or accessible to the detection reagents where the target polynucleotide is to be detected directly. Where the sample contains cells, the cells can be lysed or permeabilized to release the polynucleotides within the cells. One step permeabilization buffers can be used to lyse cells which allow further steps to be performed directly after lysis, for example a polymerase chain reaction.

### The Probe Polynucleotide

The probe polynucleotide can be RNA or DNA, and can be single-stranded, double-stranded, or a higher order polynucleotide complex and may be complexed with other molecules, for example proteins, oligosaccharides, haptens and/or lipids. The probe polynucleotide can be prepared synthetically of purified from a biological source. The probe polynucleotide may be purified to remove or diminish one or more undesired components of the sample or to concentrate the probe polynucleotide. Conversely, where the probe polynucleotide is too concentrated for the particular assay, the probe polynucleotide may be diluted.

Following sample collection and optional nucleic acid extraction, the nucleic acid portion of the sample comprising the probe polynucleotide is typically subjected to one or more preparative reactions. These preparative reactions can include in vitro transcription (IVT), labeling, fragmentation, amplification and other reactions. RNA obtained from cells can first be treated with reverse transcriptase and a primer to create cDNA prior to detection and/or amplification or can be done in situ, e.g. in cells or tissues affixed to a slide. Nucleic acid amplification increases the copy number of the probe polynucleotide and can be used to incorporate the tag sequence into the probe polynucleotide using a primer comprising the tag sequence. A variety of amplification methods are suitable for use, including the polymerase chain reaction method or (PCR), the ligase chain reaction (LCR), self sustained sequence replication (3SR), nucleic acid sequence-based amplification (NASBA), the use of Q Beta replicase, reverse transcription, nick translation, and the like.

The probe polynucleotide is typically amplified by contacting one or more strands of the probe polynucleotide with a primer and a polymerase having suitable activity to extend the primer and copy the probe polynucleotide to produce a full-length complementary polynucleotide or a smaller portion thereof. Any enzyme having a polymerase activity which can copy the probe polynucleotide can be used, including DNA polymerases, RNA polymerases, reverse transcriptases, enzymes having more than one type of polymerase activity, and can be thermolabile or thermostable. Mixtures of enzymes can also be used. Exemplary enzymes include: DNA polymerases such as DNA Polymerase I (“Pol I”), the Klenow fragment of Pol I, T4, T7, Sequenase® T7, Sequenase® Version 2.0 T7, Tub, Taq, Tth, Pfx, Pfu, Tsp, Tfl, Tli and Pyrococcus sp GB-D DNA polymerases; RNA polymerases such as E. coli, SP6, T3 and T7 RNA polymerases; and reverse transcriptases such as AMV, M-MuLV, MMLV, RNAse MMLV (SuperScript®), SuperScript® II, ThermoScript®, HIV-1, and RAV2 reverse transcriptases. All of these enzymes are commercially available. Exemplary polymerases with multiple specificities include RAV2, and Tli (exo-) polymerases. Exemplary thermostable polymerases include Tub, Taq, Tth, Pfx, Pfu, Tsp, Tli and Pyrococcus sp. GB-D DNA polymerases.

Suitable reaction conditions are chosen to permit amplification of the probe polynucleotide, including pH, buffer, ionic strength, presence and concentration of one or more salts, presence and concentration of reactants and cofactors such as nucleotides and magnesium and/or other metal ions, optional cosolvents, temperature, thermal cycling profile for amplification schemes comprising a polymerase chain reaction, and may depend in part on the polymerase being used as well as the nature of the sample. Cosolvents include formamide (typically at from about 2 to about 10%), glycerol (typically at from about 5 to about 10%), and DMSO (typically at from about 0.9 to about 10%). Techniques may be used in the amplification scheme in order to minimize the production of false positives or artifacts produced during amplification. These include “touchdown” PCR, hot-start techniques, use of nested primers, or designing PCR primers so that they form stem-loop structures in the event of primer-dimer formation and thus are not amplified. Techniques to accelerate PCR reactions can be used, for example centrifugal PCR, which allows for greater convection within the sample, and comprising infrared heating steps for rapid heating and cooling of the sample.

The tag sequence is a polynucleotide sequence which is not present in the biological source of the probe polynucleotide and can bind to a corresponding tag-binding polynucleotide under assay conditions. It can be incorporated into the probe polynucleotide at or nearer either the 3′ end or the 5′ end through a variety of techniques, for example ligation to either end of a polynucleotide, addition by terminal transferase, or through incorporation in a primer used to prime an amplification reaction forming the primer. Where the probe polynucleotide is prepared synthetically, the tag sequence can be incorporated during the synthesis. Typically the tag sequence is introduced at or nearer the 5′ end by incorporation into a primer used to prime either a reverse transcription reaction or a polymerase chain reaction. The primer can hybridize to a probe-specific sequence in the sample, or may contain a poly (dT) sequence used to prime reverse transcription of all the mRNAs in the sample. A plurality of different primers may be used to amplify different regions of a particular polynucleotide within the sample, and these different primers may contain the same tag sequence or may contain different tag sequences. The primers may incorporate bases or mixtures of bases which allow the primer or mixture of primers thus resulting to prime syntheses from a plurality of sequences within the sample; this may be accomplished by synthesizing the primer to have bases which base-pair with more than one complementary nucleotide at the 3′ end of the primer, or by synthesizing a primer mixture having different nucleotides represented at the 3′ end of the primers within the mixture; this can occur at the ultimate 3′ base, the penultimate 3′ base, the four most 3′ bases, the six most 3′ bases, etc., depending on the degeneracy which is desired in the primer binding.

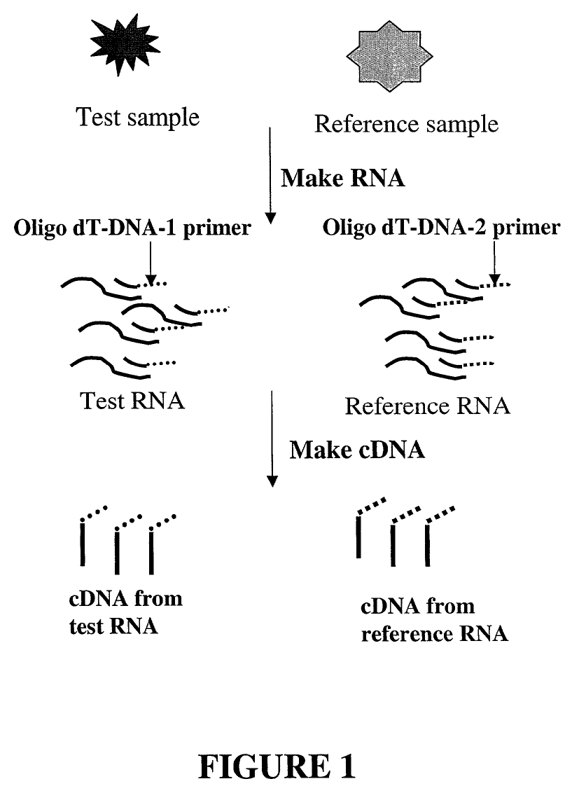

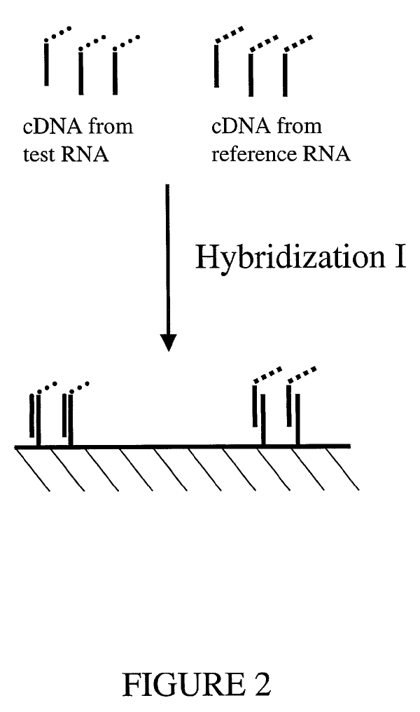

For example, the preparation of tagged probe polynucleotides from RNA in the sample by reverse transcription can be performed by using methods described in Khan et al. (1999) Biochim. Biophys. Acta 142317-28 or from www.nhgri.nih.gov/DIR/microarray or other published methods. A generic sequence tag is added to the 5′ end of the oligo (dT) primer for reverse transcription with equal amount of unmodified dNTPs. The resulting cDNA is labeled with a generic sequence tag at the 5′ end. For two-color microarray experiments, test cDNA can be labeled with a first tag and reference cDNA can be labeled with a second tag (FIGS. 1 and 2). For more than two colors, different test cDNA can be conjugated with additional different tags or with different haptens (e.g., biotin) that can be linked to different tags.

Amplified probe polynucleotides may be subjected to post amplification treatments. For example, in some cases, it may be desirable to fragment the probe polynucleotide prior to hybridization with an polynucleotide array, in order to provide segments which are more readily accessible to the probes, which avoid looping and/or hybridization to multiple probes. Fragmentation of the nucleic acids may generally be carried out by physical, chemical or enzymatic methods known in the art.

### Tag-Binding Conjugates

The present invention uses a composition comprising SCNCs conjugated to a tag-binding polynucleotide, such that the composition can be used to detect the presence and/or amount of the probe polynucleotide(s) in the sample via the methods disclosed herein. The tag-binding polynucleotide can bind to the tag sequence on the probe polynucleotide under suitable hybridization conditions.

Any technique can be used for linking SCNCs to the tag-binding polynucleotide which allows the tag-binding polynucleotide to bind to the tag sequence of the probe polynucleotide. Suitable techniques are known in the art.

The surface layer of the SCNCs may be modified by displacement to render the SCNC reactive for a particular coupling reaction. For example, displacement of trioctylphosphine oxide (TOPO) moieties with a group containing a carboxylic acid moiety enables the reaction of the modified SCNCs with amine containing moieties to provide an amide linkage. For a detailed description of these linking reactions, see, e.g., U.S. Pat. No. 5,990,479; Bruchez et al. (1998) Science 281:2013-2016, Chan et al. (1998) Science 281:2016-2018, Bruchez “Luminescent SCNCs: Intermittent Behavior and use as Fluorescent Biological Probes” (1998) Doctoral dissertation, University of California, Berkeley, and Mikulec “SCNC Colloids: Manganese Doped Cadmium Selenide, (Core) Shell Composites for Biological Labeling, and Highly Fluorescent Cadmium Telluride” (1999) Doctoral dissertation, Massachusetts Institute of Technology. The SCNC may be conjugated directly to the tag-binding polynucleotide or indirectly through a linker moiety.

Examples of suitable spacers or linkers are polyethyleneglycols, dicarboxylic acids, polyamines and alkylenes, optionally substituted with functional groups, for example hydrophilic groups such as amines, carboxylic acids and alcohols or lower carbon alcohols such as methoxy and ethoxy groups. Additionally, the spacers will have an active site on or near a distal end. The active sites are optionally protected initially by protecting groups. Among a wide variety of protecting groups which are useful are FMOC, BOC, t-butyl esters, t-butyl ethers, and the like. Various exemplary protecting groups are described in, for example, Atherton et al., Solid Phase Peptide Synthesis, IRL Press (1989).

### Production of SCNCs

SCNCs for use in the subject methods can be made from any material and by any technique that produces SCNCs having emission characteristics useful in the methods, articles and compositions taught herein. Suitable methods of production are disclosed in U.S. Pats. Nos. 6,048,616; 5,990,479; 5,690,807; 5,505,928; 5,262,357, as well as PCT Publication No. WO 99/26299 (published May 27, 1999).

The SCNCs have absorption and emission spectra that depend on their size, size distribution and composition. These SCNCs can be prepared as described in Murray et al. (1993) J. Am. Chem. Soc. 115:8706-8715, Guzelian et al. (1996) J. Phys. Chem. 100:7212-7219 or PCT Publ. No. WO 99/26299 (inventors Bawendi et al.).

Examples of materials from which SCNCs can be formed include group II-VI, III-V and group IV semiconductors such as ZnS, ZnSe, ZnTe, CdS, CdSe, CdTe, MgS, MgSe, MgTe, CaS, CaSe, CaTe, SrS, SrSe, SrTe, BaS, BaSe, BaTe, GaN, GaP, GaAs, GaSb, InP, InAs, InSb, AlS, AlP, AlSb, PbS, PbSe, Ge, Si, and ternary and quaternary mixtures thereof.

The composition, size and size distribution of the semiconductor nanocrystal affect its absorption and emission spectra. Exemplary SCNCs that emit energy in the visible range include CdS, CdSe, CdTe, ZnSe, ZnTe, GaP, and GaAs. Exemplary SCNCs that emit energy in the near IR range include InP, InAs, InSb, PbS, and PbSe. Exemplary SCNCs that emit energy in the blue to near-ultraviolet include ZnS and GaN. The size of SCNCs in a given population can be determined by the synthetic scheme used and/or through use of separation schemes, including for example size-selective precipitation and/or centrifugation. The separation schemes can be employed at an intermediate step in the synthetic scheme or after synthesis has been completed. For a given composition, larger SCNCs absorb and emit light at longer wavelengths than smaller SCNCs. SCNCs absorb strongly in the visible and UV and can be excited efficiently at wavelengths shorter than their emission peak. This characteristic allows the use in a mixed population of SCNCs of a single excitation source to excite all the SCNCs if the source has a shorter wavelength than the shortest SCNC emission wavelength within the mixture; it also confers the ability to selectively excite subpopulation(s) of SCNCs within the mixture by judicious choice of excitation wavelength.

The surface of the SCNC is preferably modified to enhance emission efficiency by adding an overcoating layer to form a “shell” around the “core” SCNC, because defects in the surface of the core SCNC can trap electrons or holes and degrade its electrical and optical properties. Addition of an insulating shell layer eliminates nonradiative relaxation pathways from the excited core, resulting in higher luminescence efficiency. Suitable materials for the shell include semiconductor materials having a higher bandgap energy than the core and preferably also having good conductance and valence band offset. Thus, the conductance band of the shell is desirably of a higher energy and the valence band is desirably of a lower energy than those of the core. For SCNC cores that emit energy in the visible (e.g., CdS, CdSe, CdTe, ZnSe, ZnTe, GaP, GaAs) or near IR (e.g., InP, InAs, InSb, PbS, PbSe), a material that has a bandgap energy in the ultraviolet may be used for the shell, for example ZnS, GaN, and magnesium chalcogenides, e.g., MgS, MgSe, and MgTe. For an SCNC core that emits in the near IR, materials having a bandgap energy in the visible, such as CdS or CdSe, or the ultraviolet may be used. Preparation of core-shell SCNCs is described in, e.g., Dabbousi et al. (1997) J. Phys. Chem. B 101:9463; Kuno et al., J. Phys. Chem. 106:9869 (1997); Hines et al., J. Phys. Chem. 100:468; and PCT Publ. No. WO 99/26299. The SCNCs can be made further luminescent through overcoating procedures as described in Danek et al. (1996) Chem. Mat. 8(1):173-180, Peng et al. (1997) J. Am. Chem. Soc. 119:7019-7029.

Most SCNCs are typically prepared in coordinating solvent, such as TOPO and trioctyl phosphine (TOP), resulting in the formation of a passivating organic layer on the surface of SCNCs with and without a shell. Such passivated SCNCs can be readily solubilized in organic solvents, for example toluene, chloroform and hexane. Molecules in the passivating layer can be displaced or modified to provide an outermost coating that adapts the SCNCs for use in other solvent systems, for example aqueous systems. Furthermore, based upon the desired application, a portion of the SCNC functionality, or the entire surface of the SCNC functionality may be modified by a displacement reaction, based upon the desired use therefor. Preferred molecules used in such a displacement reaction are those described in U.S. App. Ser. No. 60/240,216, filed Oct. 13, 2000 by Adams et al. entitled “Hydrophobically Modified Water-Soluble Polymers And Polypeptides As Dispersants For Hydrophobic Semiconductor And Metal Nanoparticles.”

Alternatively, an outermost layer of an inorganic material such as silica can be added around the shell to improve the aqueous dispersibility of the SCNCs, and the surface of the silica can optionally be derivatized (Bruchez et al., Science 281:2013 (1998)).

A displacement reaction may also be employed to modify the SCNC to improve the solubility in a particular organic solvent. For example, if it is desired to associate the SCNCs with a particular solvent or liquid, such as pyridine, the surface can be specifically modified with pyridine or pyridine-like moieties which are soluble or miscible with pyridine to ensure solvation. Water-dispersible SCNCs can be prepared as described in Bawendi et al., PCT Publ. No. WO 00/17655, published Mar. 30, 2000.

### Detection of SCNC Emission

An example of an imaging system for automated detection for use with the present methods comprises an excitation source, a monochromator (or any device capable of spectrally resolving the image, or a set of narrow band filters) and a detector array. The excitation source can comprise a blue or UV source of light, of a wavelength shorter than that of the luminescence to be detected. This may be a broadband IN light source, such as a deuterium lamp with a filter in front; the output of a white light source such as a xenon lamp or a deuterium lamp after passing through a monochromator to extract out the desired wavelengths; or any of a number of continuous wave (cw) gas lasers, including but not limited to any of the Argon Ion laser lines (457, 488, 514, etc. nm), a HeCd laser; solid state diode lasers in the blue such as GaN and GaAs (doubled) based lasers or the doubled or tripled output of YAG or YLF based lasers; or any of the pulsed lasers with output in the blue.

The emitted light can be detected with a device that provides spectral information for the substrate, e.g., grating spectrometer, prism spectrometer, imaging spectrometer, or the like, or use of interference (bandpass) filters. Using a two-dimensional area imager such as a CCD camera, many objects may be imaged simultaneously. Spectral information can be generated by collecting more than one image via different bandpass, longpass, or shortpass filters (interference filters, or electronically tunable filters are appropriate). More than one imager may be used to gather data simultaneously through dedicated filters, or the filter may be changed in front of a single imager. Imaging based systems, like the Biometric Imagn system, scan a surface to find fluorescent signals.

A scanning system can be used in which the sample to be analyzed is scanned with respect to a microscope objective. The luminescence is put through a single monochromator or a grating or prism to spectrally resolve the colors. The detector is a diode array that then records the colors that are emitted at a particular spatial position. The software then recreates the scanned image.

Use of the Method with DNA Microarrays

cDNA microarray slides for expression profiling can be prepared as described in the Fabrication section of www.nhgri.nih.gov/DIR/microarray. Further information on fabrication, sample labeling and conditions for hybridization using microarrays is provided, for example, by Bittner et al. (2000) Nature 406:536-540, Khan et al. (1999) Electrophoresis 20:223-9, Duggan (1999) Science 283:83-87, and DeRisi et al. (1996) Nature Genet. 14:457-60.

Microarrays spotted with DNA for genotyping can be purchased from Operon Technologies, Inc. or from other sources.

In a typical microarray experiment, total RNA is extracted from test and reference (normal or control) cells. The test and reference RNAs are reverse transcribed using different primers comprising different tag sequences followed by dTs. The unincorporated primers can optionally be removed using commercial available PCR purification kits. The probes are combined, optionally mixed with blockers, for example tRNA, Cot1 DNA, or purified repeat sequences such as LINE or Alu sequences, or mixtures thereof. The probe mixture is first hybridized to the targets on microarray slides (for detection of SNPs or gene expression). The excess probes are removed and the slide is hybridized again with corresponding tag-binding conjugates. After hybridization the excess conjugates are removed and the slides scanned.

In one embodiment, a hybridization mixture can be prepared from a sample containing or suspected of containing tagged probe polynucleotide cDNAs in 4×SSC and 1 g/ml poly dA (Pharmacia). This hybridization mixture can be denatured at 98° C. for 2 minutes and cooled to 45° C. A small volume of 10% SDS solution is added to bring the final concentration to 0.2% SDS. 15-30 uL of this hybridization mixture can be applied to a typical size microarray, which are then covered with a coverslip and placed in a humidified chamber overnight at 65° C. to hybridize. After hybridization, the microarray slide is sequentially rinsed in 1×SSC with 0.03% SDS, 0.2×SSC and 0.05×SSC. The slide is spin-dried in a centrifuge with a horizontal rotor at 500 rpm for 5 minutes. The slide is incubated in blocking solution (4×SSC, 0.1% Tween® 20, 1% bovine serum albumin (“BSA”)) for 30-60 minutes at room temperature, rinsed in 0.05×SSC, and dried by spinning in a centrifuge.

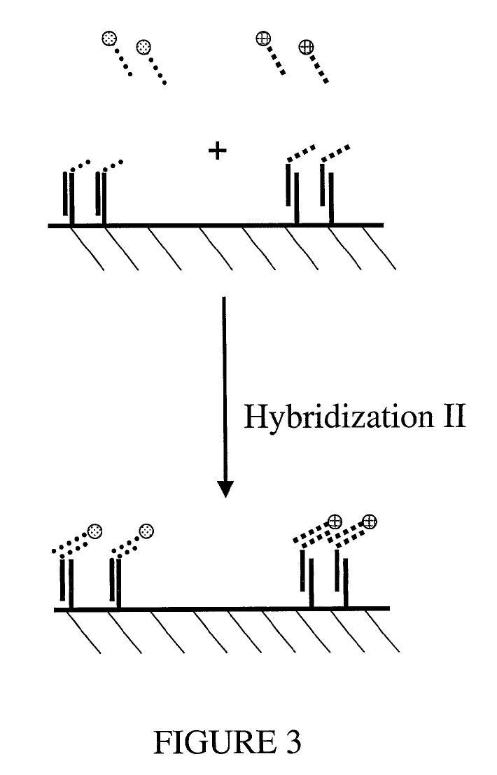

First and second tag-binding conjugates comprising first and second semiconductor nanocrystals, respectively, can be added to a final concentration of approximately 100 nM each in 4×SSPE, 1% BSA (FIG. 3) to form a second hybridization mixture. 40-80 uL of this second mixture is then applied on a typical size array and covered with a glass coverslip, and the slide is then incubated in a humidified container for 1 hour at 30-45° C. The slide is rinsed in 1×SSPE, 0.03% SDS followed by 0.06×SSPE and dried by spinning in centrifuge at 500 rpm for 5 minutes. The microarray can then be scanned with a laser scanner having an excitation source and emission filters appropriate for the particular SCNC(s) used, or the microarray can be scanned with a wide-field imaging scanner having appropriate excitation and emission filters for the SCNC(s) used.

### Use of the Method in Fluorescence In Situ Hybridization

In a typical fluorescence in-situ hybridization (FISH) experiment, probes for FISH can be made by PCR using standard methods (e.g. random or targeted PCR priming on a DNA of interest using one or more pairs of PCR primers to amplify one or more different regions of the DNA of interest), see, e.g., PCT Publ. No. WO 00/68692 to Bruchez et al., published Nov. 16, 2000, and references cited therein. Different tag sequences can be incorporated into the PCR primers for different probe polynucleotides. Different probes are combined, and optionally mixed with blockers as described above. The probe mixture is hybridized to the target slides for FISH (DNA or RNA). The excess probes are removed and the slide is then hybridized for a second time corresponding tag-binding conjugates. After hybridization, the excess tag-binding conjugates are removed and the slides scanned.

### Kits

Kits comprising reagents useful for performing the methods of the invention are also provided. In one embodiment, a kit comprises: (1) a substrate comprising a first target polynucleotide and (2) a first tag-binding conjugate comprising a first semiconductor nanocrystal and a first tag-binding polynucleotide, each of which is retained by a housing. A probe polynucleotide is required for binding of the first tag-binding conjugate to the first target polynucleotide, and a sample may be assayed for the presence of such a probe polynucleotide using the components of the kit. Instructions for using the kit to perform a method of the invention are provided with the housing, and may be located inside the housing or outside the housing, and may be printed on the interior or exterior of any surface forming the housing which renders the instructions legible. A tag polynucleotide can optionally be included in the kit for preparation of the probe polynucleotide. The kit may be in multiplex form, containing pluralities of one or more different substrates, target polynucleotides, tag-binding conjugates, and/or tag polynucleotides. The substrate may be a microarray, or may be a plurality of different beads wherein each of the different beads comprises a different target polynucleotide.

### EXAMPLES

The following examples are set forth so as to provide those of ordinary skill in the art with a complete description of how to make and use the present invention, and are not intended to limit the scope of what is regarded as the invention. Efforts have been made to ensure accuracy with respect to numbers used (e.g., amounts, temperature, etc.) but some experimental error and deviation should be accounted for. Unless otherwise indicated, parts are parts by weight, temperature is degree centigrade and pressure is at or near atmospheric, and all materials are commercially available.

The sequences of the oligonucleotides used in the following examples are set forth in Table 1:

TABLE 1

| Oligo | | Genbank | Gene | SEQ |
|---|---|---|---|---|
| name | | accession | name | ID NO: |
| | Oligo 70mer sequence | | | |
| | (L = NH2) | | | |
| h-136 | LTTGAGCAGTGGGCTCACTCTGAAGA | U56390 | Caspase 9 | |
| | CCTGCAGTCCCTCCTGCTTAGGGTCG | | | |
| | CTAATGCTGTTTCGGTGAA | | | |
| h-252 | LCCGCGCCGACAAACAGAACCTGGAG | AE041835 | Laminin γ3 | |
| | GCCATTCTGCACAGCCTGCCCGAGAA | | precursor; | |
| | CTGTGCCAGCTGGCAGTGA | | LAMC3 | |
| h-501-b | LGCTCCCAGAATTTCAGCTTCAGCTT | K00558 | Alpha- | |
| | AACTGACAGATGTTAAAGCTTTCTGG | | tubulin | |
| | TTAGATTGTTTTCACTTGG | | | |
| h-503-b | LCCACCTGTCCCTCCTGGGCTGCTGG | U14971 | Ribosomal | |
| | ATTGTCTCGTTTTCCTGCCAAATAAA | | protein S9 | |
| | CAGGATCAGCGCTTTAAAA | | | |
| | 50mer complement with biotin | | | |
| | at 5′ end (X = biotin) | | | |
| h-136r50 | XTTCACCGAAACAGCATTAGCGACCC | | | |
| | TAAGCAGGAGGGACTGCAGGTCTTC | | | |
| h-252r50 | XTCACTGCCAGCTGGCACAGTTCTCG | | | |
| | GGCAGGCTGTGCAGAATGGCCTCCA | | | |
| h-501- | XCCAAGTGAAAACAATCTAACCAGA | | | |
| br50 | AAGCTTTAACATCTGTCAGTTAAGCT | | | |
| h-503- | XTTTTAAAGCGCTGATCCTGTTTATT | | | |
| br50 | TGGCAGGAAAACGAGACAATCCAGC | | | |
| | Complement oligo with | | | |
| | generic tag | | | |
| c186-h- | G GCG TGG CGG GGA AAG CAT | | | |
| 136 | TTCACCGAAACAGCATTAGCGACCCT | | | |
| | AAGCAGGAGGGACTGCAGGTCTTC | | | |
| clambda- | GGG CGG CGA CCT T | | | |
| h-136 | TTCACCGAAACAGCATTAGCGACCCT | | | |
| | AAGCAGGAGGGACTGCAGGTCTTC | | | |
| clambda- | GGG CGG CGA CCT T | | | |
| h-252 | TCACTGCCAGCTGGCACAGTTCTCGG | | | |
| | GCAGGCTGTGCAGAATGGCCTCCA | | | |
| c186-h- | G GCG TGG CGG GGA AAG CAT | | | |
| 252 | TCACTGCCAGCTGGCACAGTTCTCGG | | | |
| | GCAGGCTGTGCAGAATGGCCTCCA | | | |
| | Oligos for attachment | | | |
| | to SCNCs | | | |
| B-lambda | 5′-Biotin-CTG GAA CAA CAC TCA | | | |
| CATCH | CAA GGT CGC CGC CC-3′ | | | |
| B-186 | 5′-Biotin-CTG GAA CAA CAC TCA | | | |
| CATCH | CAA TGC TTT CCC CGC CAC GCC-3′ | | | |

It is to be understood that while the invention has been described in conjunction with the preferred specific embodiments thereof, the foregoing description as well as the examples which follow are intended to illustrate and not limit the scope of the invention. Other aspects, advantages and modifications within the scope of the invention will be apparent to those skilled in the art to which the invention pertains.

### Example 1

### Preparation of Tag-Binding Conjugates

A conjugate of an SCNC having an emission maximum of 520 nM (“520 nm SCNC”) and the clambda polynucleotide and a conjugate of an SCNC having an emission maximum of 630 nM (“630 nm SCNC”) and the c186 polynucleotide were prepared by continuously mixing 520 nM SCNC-streptavidin with a biotinylated B-lambda CATCH polynucleotide and 630 nM SCNC-streptavidin with biotinylated B-186 CATCH for approximately 1-2 hours at room temperature to indirectly link the SCNCs with the tag-binding polynucleotides. The ratio of biotinylated DNA to SCNC-streptavidin was 1:2.

### Example 2

### Microarrays Comprising Target Polynucleotides

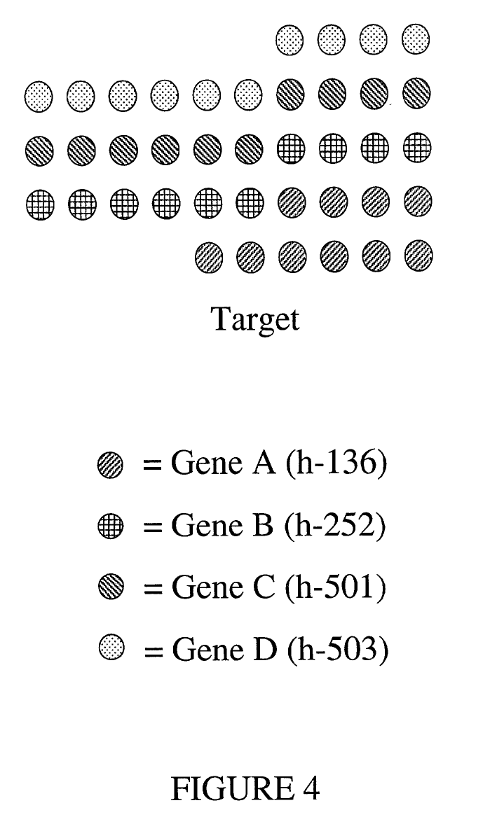

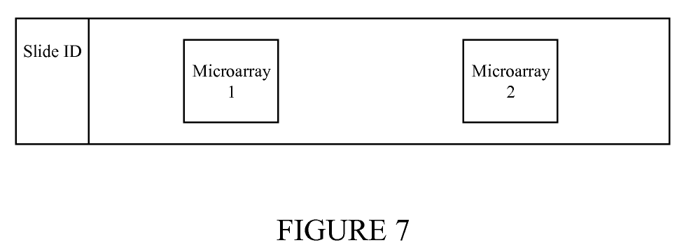

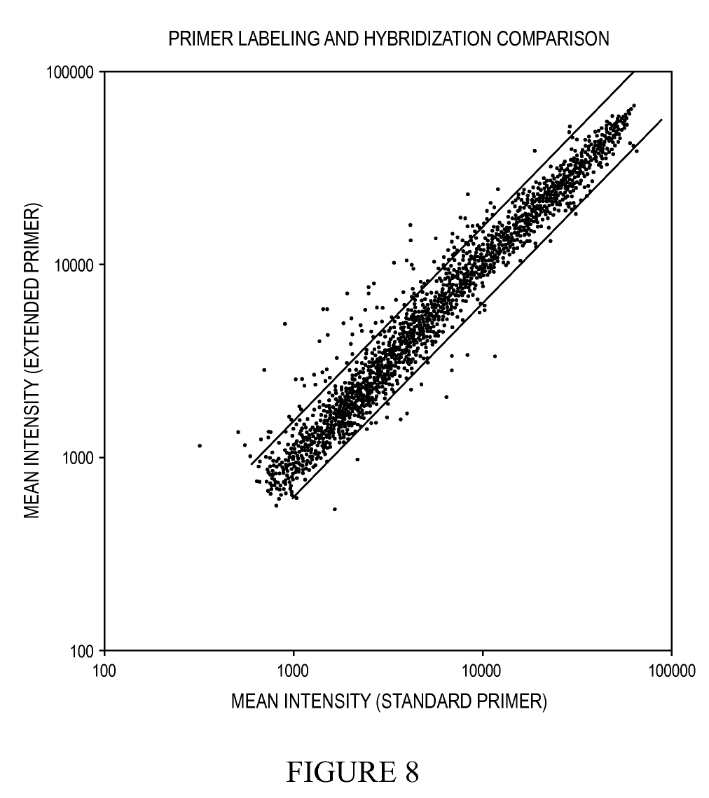

Microarray slides printed with DNA targets were purchased from Operon Technologies, Inc. (FIG. 7). Each slide has two printed areas (2 microarrays) containing four target genes (gene A=h-136, gene B=h-252, gene C=h-501-b, and gene D=h-503b) spotted 10 times each (FIG. 4). The sequences of the DNA targets, the biotinylated 50-mer polynucleotides complementary to h-136, h-252, h-501 and h-503b, the complementary DNAs to the h-136 and h-252 genes with generic sequence tags at the 5′ end, and the tag-binding oligonucleotides for attachment to SCNCs are provided in Table 1. All DNA molecules were purchased from Operon.

### Example 3

### Two-Color Microarray Labeling Using SCNC-DNA Conjugates

The following experiment demonstrates the use of two different SCNC-DNA conjugates having different emission wavelengths to bind to tag sequences on probe polynucleotides bound to target polynucleotides arrayed on a substrate. 30 uL of hybridization buffer mix I containing 166 nM each of the DNAs clambda-h-136, c186-h-136, and 333 nM c186-h-252 in 4×SSC along with 16 ug poly dA (Pharmacia), 8 ug yeast tRNA (Sigma), and 180 ug herring sperm DNA as carrier was prepared as a probe mixture. The probe mixture was denatured at 98° C. for 2 minutes, cooled to 45° C. and a small volume of 10% SDS solution was added to a final concentration of 0.2% SDS. The probe mixture was applied onto the slide (two microarray areas), covered with a cover slip, placed in a humidified chamber and incubated overnight at 65° C. After hybridization, the slide was sequentially rinsed in 1×SSC with 0.03% SDS, 0.2× SSC and 0.05×SSC. The slide was spun dried in centrifuge with horizontal rotor at 500 rpm for minutes. The slide was then incubated in blocking solution 4×SSC, 0.1% Tween® 20, 1% BSA for 30-60 minutes at room temperature, rinsed in 0.05×SSC, and dried by spinning in a centrifuge.

A second hybridization mix (15 uL) containing 100 nM each of 520 nM SCNC-clambda and 630 nM SCNC-c186 in 4×SSPE, 1% BSA, along with 4 ug yeast tRNA and 90 ug herring sperm DNA as carrier was prepared. This second mix was applied onto one of the microarray areas, covered with a coverslip and incubated in a humidified container for 1 hour at 35° C. The slide was then rinsed in 1×SSPE, 0.03% SDS followed by 0.06×SSPE and dried by spinning in a centrifuge at 500 rpm for 5 minutes. The microarray was then read in a microarray scanner.

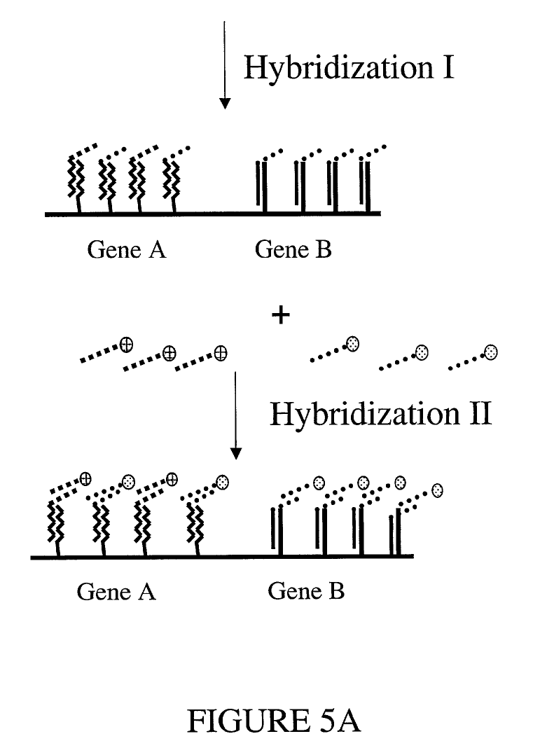

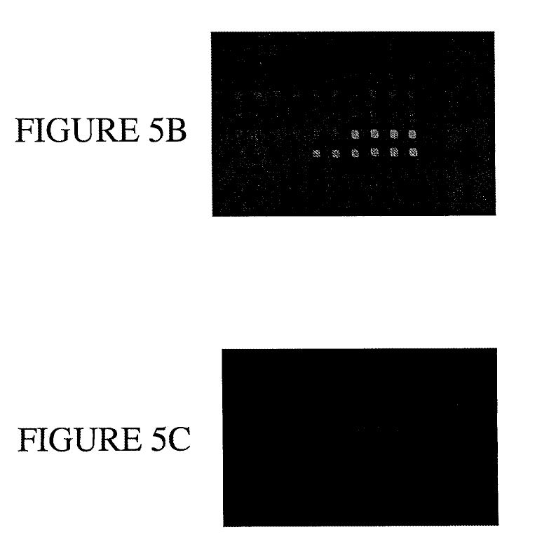

Results of scanning showed that the 520 nM SCNC-clambda (green) hybridized to gene h-136 (gene A) and 630 nM SCNC-c186 (red) hybridized to both gene h-136 (gene A) and h-252 (gene B) (FIG. 5A-C).

### Example 4

### Three-Color Microarray Labeling Using SCNC-DNA and SCNC-Hapten Conjugates

The following experiment demonstrates the use of two different SCNC-DNA conjugates having different emission wavelengths and one SCNC-labeled hapten to bind to tag sequences on probe polynucleotides bound to target polynucleotides arrayed on a substrate. 30 uL of hybridization buffer mix I containing 333 nM of the tagged DNAs clambda-h-136, c186-h-136, clambda-h-252, c186-h-252 and h-136r50 in 4×SSC along with 16 ug poly dA (Pharmacia), 8 ug yeast tRNA (Sigma), and 180 ug herring sperm DNA as carrier was prepared as a probe mixture. The probe mixture was denatured at 98° C. for 2 minutes, cooled to 45° C. and a small volume of 10% SDS solution was added to a final concentration of 0.2% SDS. The probe mix was applied onto the two microarray areas on the slide, covered with a coverslip, placed in a humidified chamber and incubated overnight at 65° C. After hybridization, the slide was sequentially rinsed in 1×SSC with 0.03% SDS, 0.2×SSC, 0.05×SSC and spun dried in centrifuge with horizontal rotor at 500 rpm for 5 minutes. The slide was then incubated in blocking solution 4×SSC, 0.1% Tween® 20, 1% BSA for 30-60 minutes at room temperature, rinsed in 0.05×SSC, and dried by spinning in centrifuge. The slide was incubated with approximately 33 nM of 592 nm SCNC-streptavidin conjugate in 6×SSPE, 1% BSA, 10 mM MgCl₂ for 1 hour at room temperature. The slide was rinsed with 0.05×SSPE and spun dried in centrifuge with horizontal rotor at 500 rpm for 5 minutes.

A second hybridization mix II (15 uL) containing approximately 200 nM each of 520 nM SCNC-c186 and 630 nM SCNC-clambda in 4×SSPE, 1% BSA, along with 4 ug yeast tRNA and 90 ug herring sperm DNA as carrier was prepared. This second mix was applied onto one of the microarray areas, a coverslip was added and the slide was incubated in a humidified container for 1 hour at 35° C. The slide was then rinsed in 1×SSPE, 0.03% SDS followed by 0.06×SSPE and dried by spinning in centrifuge at 500 rpm for 5 minutes. The microarray was then read in a microarray scanner.

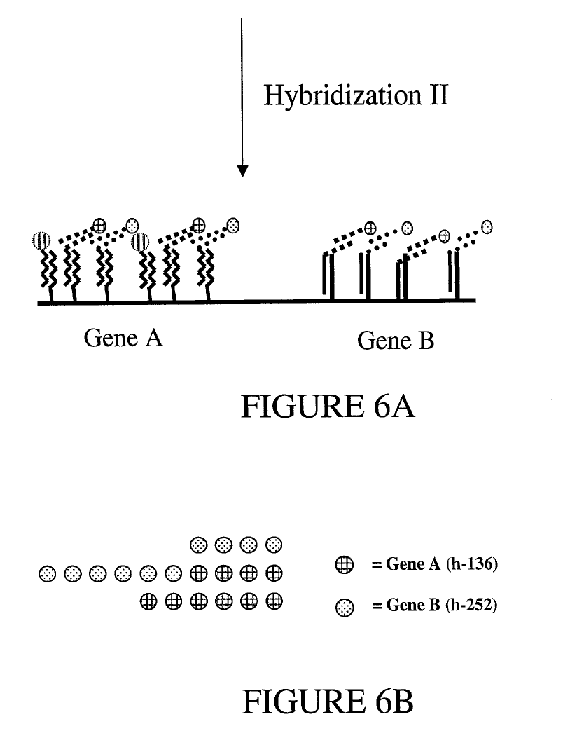

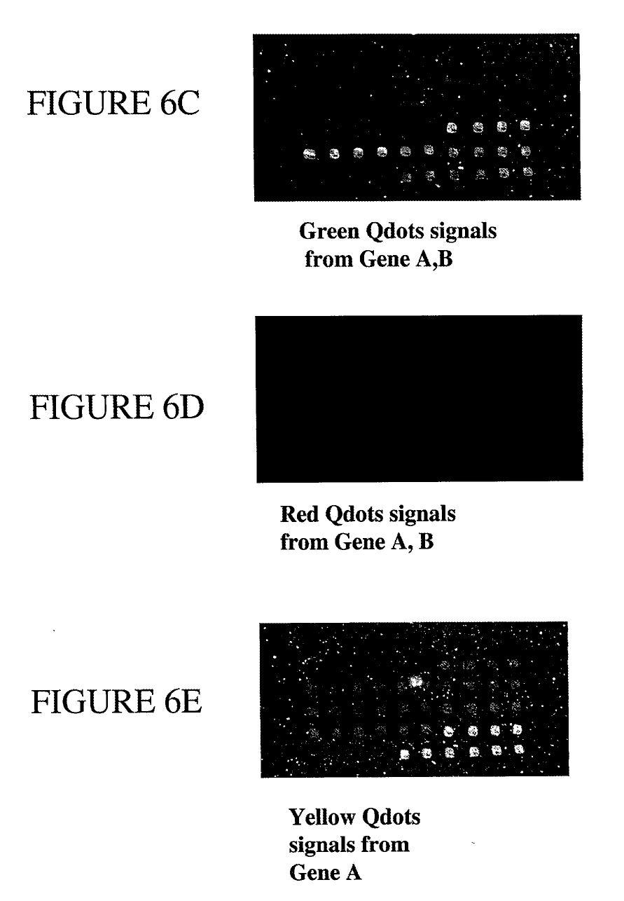

Results of scanning showed that the 592 nm SCNC-streptavidin (yellow) binds to the biotinylated DNA h-136-br50 (gene A), 520 nm SCNC-c186 (green) hybridized to gene h-136 (gene A), h-252 (gene B) and 630 nm SCNC-clambda (red) hybridized to both gene h-136 (gene A) and h-252 (gene B) (FIG. 6A-D).

### Example 5

### Tag Sequences do not Inhibit Probe Binding to Target

This experiment demonstrates that detection of polynucleotides on an array using probe polynucleotides comprising a tag sequence produces equivalent results to probe polynucleotides lacking a tag. A reverse transcription primer (RT186) was designed. This sequence of this primer is 5′-GGCGTGGCGGGGAAAGCATTTTTTTTTTTTTTTTTTTTTVN-3′. The 5′ extension of the primer is identical to one of the strands of the bacteriophage 186 cos site. Such sites serve the purpose of forming hybrids at physiological temperatures, driving circularization of phage genomes in their host cells. The inclusion of a 5′ extension readily able to hybridize to its complement at modest temperatures (25-37 C) is intended to allow a two hybridization approach to quantitating mRNA transcripts with microarrays. In the first step, cDNAs synthesized from sample RNAs using the extended primer are hybridized in a high temperature, stringent hybridization to a microarray. After the first hybridization, non-hybridized strands are removed and then a second, low-temperature hybridization is carried out with semiconductor nanocrystals derivatized with oligonucleotides that are the complement of the 5′ extension of the hybridized strands. One requirement for this approach to work is that the 5′ extension not cause spurious hybridization to the immobilized cDNAs during the first, stringent hybridization.

This was tested explicitly by comparing the hybridization of cDNAs synthesized from a single pool of RNA. Microarray fabrication, RNA isolation, labeling and hybridization were carried out according to publicly available protocols (http://www.nhgri.nih.gov/DIR/Microarray/Protocols.pdf), also set forth in U.S. patent application Ser. No. 09/675,528 by Empedocles et al. entitled “Microarray Methods Utilizing Semiconductor Nanocrystals”, filed Sep. 29, 2000. Further guidance on fabrication, sample labeling and conditions for hybridization using microarrays is provided, for example, by Bittner M., et al. (2000) Nature 406:536-540; Khan J., et al. (1999) Electrophoresis 20:223-9; Duggan, D. J. (1999) Science 283:83-87; and DeRisi, J. et al. (1996) 14:457-60.

RNA was isolated from the cell line UACC903, a melanoma cell line from the University of Arizona Cancer Center. Two separate species of fluorescently labeled cDNA were prepared from the UACC903 RNA. One species resulted from reverse transcription of 100 micrograms of total RNA, using two micrograms of RT186 primer and the fluorescently labeled nucleotide Cy3-dUTP (Amersham Pharmacia). The other species resulted from reverse transcription of 200 micrograms of total RNA, using two micrograms of standard dT (12-18) primer and the fluorescently labeled nucleotide Cy5-dUTP (Amersham Pharmacia). The two species were combined and hybridized to a microarray on which were printed PCR amplified cDNA products from 6144 Image Consortium Clones from the Research Genetics Human Named Gene set (Research Genetics, Huntsville Ala.), 176 Image Consortium Clones chosen as normalization controls from the Soares infant brain 1NIB EST library and 24 controls for non-specific hybridization. After washing away non-hybridized labeled cDNA, the slide was scanned on a confocal imaging system (Agilent, Palo Alto, Calif.). Image analysis to extract fluorescent intensities at each array element and normalize the data from the two fluorescence channels was carried out according to the methods described in Chen, Y., Dougherty, E. R. and Bittner, M. L. (1997), “Ratio-based decisions and the quantitative analysis of cDNA microarray images” J. Biomed Optics 2(4): 364-74.

Although the invention has been described in some detail with reference to the preferred embodiments, those of skill in the art will realize, in light of the teachings herein, that certain changes and modifications can be made without departing from the spirit and scope of the invention. Accordingly, the invention is limited only by the claims.

What is claimed is:

1. A method for assaying a first sample for a first probe comprising:
providing a substrate attached to a first target;
contacting the substrate with the first sample, wherein the first sample is suspected of comprising the first probe, wherein the first probe comprises a first probe polynucleotide comprising a first tag sequence which does not bind to the first target and a first binding sequence which does bind to the first target, and wherein contacting the substrate with the first sample takes place under conditions in which the first binding sequence can bind to the first target;
contacting the first sample with a first tag-binding conjugate, wherein said first tag-binding conjugate comprises a first semiconductor nanocrystal conjugated to a first tag-binding polynucleotide, wherein the first tag-binding polynucleotide can bind to the first tag sequence but not to the first target, and wherein contacting the first sample with the first tag-binding conjugate takes place under conditions in which the first tag-binding polynucleotide can bind to the first tag sequence; and
determining if the first semiconductor nanocrystal is associated with the substrate.

2. The method of claim 1, wherein the substrate is selected from the group consisting of a microsphere, a chip, a slide, a multiwell plate, an optical fiber, a cell, a fixed cell, a tissue, a fixed tissue, a nucleus, and a fixed nucleus.

3. The method of claim 1, wherein the substrate comprises a plurality of different targets.

4. The method of claim 3, wherein the substrate comprises a microarray.

5. The method of claim 1, wherein the first probe polynucleotide is produced from an amplification process comprising a polymerase chain reaction.

6. The method of claim 1, wherein the first probe polynucleotide is produced from an amplification process comprising contacting the sample with an enzyme having reverse transcriptase activity under conditions in which the enzyme can reverse transcribe RNA to DNA.

7. The method of claim 1, wherein the first tag sequence is incorporated into the first probe polynucleotide by employing a first primer polynucleotide that comprises the first tag sequence in an amplification process that produces the first probe polynucleotide.

8. The method of claim 7, wherein the first primer polynucleotide binds to the polyadenylate tail of mRNA.

9. The method of claim 7, wherein the first primer polynucleotide binds to a plurality of different sequences.

10. The method of claim 9, wherein the first primer polynucleotide is degenerate at the four 3′ residues and thus comprises a mixture of primers.

11. The method of claim 9, wherein the first primer polynucleotide comprises bases at the four 3′ residues that can base pair with more than one different base.

12. The method of claim 1, wherein the first tag sequence is incorporated into the first probe polynucleotide by ligating a polynucleotide that comprises the first tag sequence to a polynucleotide that comprises the first binding sequence.

13. The method of claim 1, wherein the first tag sequence is incorporated into the first probe polynucleotide by adding nucleotides that form the first tag sequence to a polynucleotide that comprises the first binding sequence using terminal transferase.

14. The method of claim 1, wherein the first tag sequence is located at or nearer the 5′ end of the first probe polynucleotide.

15. The method of claim 1, wherein the first tag sequence is located at or nearer the 3′ end of the first probe polynucleotide.

16. The method of claim 1, wherein the first tag sequence comprises a base which is not selected from the group consisting of adenine, guanine, cytosine, thymine, and uracil.

17. The method of claim 1, wherein contacting the sample with the first target takes place prior to contacting the sample with the first tag-binding conjugate.

18. The method of claim 1, wherein contacting the sample with the first target takes place subsequent to contacting the sample with the first tag-binding conjugate.

19. The method of claim 1, wherein contacting the sample with the first target takes place simultaneously with contacting the sample with the first tag-binding conjugate.

20. The method of claim 1, wherein the target is a polynucleotide is located in a metaphase spread of chromosomes.

21. The method of claim 1, wherein the target is a polynucleotide is located in an interphase nucleus.

22. The method of claim 1, wherein the target is a polynucleotide is located in a tissue affixed to the substrate.

23. The method of claim 1, wherein determining if the first semiconductor nanocrystal is associated with the first target comprises applying a light source to the substrate that can excite the semiconductor nanocrystal, and determining if a fluorescence emission from the first semiconductor nanocrystal occurs from the substrate where the target is located.

24. The method of claim 1, wherein the first semiconductor nanocrystal comprises a core selected from the group consisting of ZnS, ZnSe, ZnTe, CdS, CdSe, CdTe, HgS, HgSe, HgTe, MgTe, GaN, GaP, GaAs, GaSb, InN, InP, InAs, InSb, AlAs, AlP, AlSb, AlS, Ge, Si, Pb, an alloy thereof, and a mixture thereof.

25. The method of claim 24, wherein the core is CdSe.

26. The method of claim 1, wherein the semiconductor nanocrystal comprises a shell.

27. The method of claim 26, wherein the shell is CdS.

28. The method of claim 1, wherein the sample is assayed to determine if the first probe is present in the sample.

29. The method of claim 1, wherein the sample is assayed to determine the amount of the first probe present in the sample.

30. The method of claim 1, further comprising assaying a sample, which may be the first sample or a second sample, for a second probe comprising contacting the substrate with the sample, wherein the substrate comprises a target which may be the first target or a second target, wherein the sample is suspected of comprising the second probe, wherein the second probe comprises a second probe polynucleotide comprising a second tag sequence which does not bind to the target and a second binding sequence, which may the same as or different than the first binding sequence, which does bind to the target, and wherein contacting the substrate with the sample takes place under conditions in which the second binding sequence can bind to the target;
contacting the sample with a second tag-binding conjugate, wherein said second tag-binding conjugate comprises a semiconductor nanocrystal, which may be the same as or different than the first semiconductor nanocrystal, conjugated to a second tag-binding polynucleotide, wherein the second tag-binding polynucleotide can bind to the second tag sequence but not to the target, and wherein contacting the sample with the second tag-binding conjugate takes place under conditions in which the first tag-binding polynucleotide can bind to the second tag sequence; and
determining if the semiconductor nanocrystal is associated with the substrate.

31. The method of claim 1, wherein the substrate is washed prior to determining if the first semiconductor nanocrystal is associated with the first target.

32. The method of claim 1, wherein a medium is added to the substrate to dilute the concentration of the first semiconductor nanocrystal prior to determining if the semiconductor nanocrystal is associated with the first target.

33. The method of claim 1, wherein a plurality of different targets are attached to the substrate, wherein each of the different targets can preferentially bind to a corresponding different probe polynucleotide, wherein the binding of each of the different targets to its corresponding different probe polynucleotide can be separately determined.

34. The method of claim 33, wherein the binding of each different probe polynucleotide to its corresponding different target can be separately determined through a different identified position at which each different target is attached to the substrate.

35. The method of claim 33, wherein the binding of each different probe polynucleotide to its corresponding different target can be separately determined through the use of a different tag-binding conjugate that binds to each different probe polynucleotide, wherein each different tag-binding conjugate comprises a different semiconductor nanocrystal with different fluorescence characteristics and wherein the hybridization of each different first probe to its corresponding different probe polynucleotide can be separately determined by determining if each different semiconductor nanocrystal is associated with the substrate.

36. The method of claim 33, wherein the hybridization of each different probe polynucleotide to its corresponding different target can be separately determined by the conditions under which it hybridizes.

37. The method of claim 33, wherein each different probe polynucleotide is bound to a different tag-binding conjugate which comprises a semiconductor nanocrystal the same as the first semiconductor nanocrystal.

38. The method of claim 33, wherein each different probe polynucleotide is bound to a different tag-binding conjugate which comprises a different semiconductor nanocrystal.

39. A first probe polynucleotide assay complex comprising a substrate attached to a first target, wherein the first target is a polynucleotide and is bound to a first binding sequence of a first probe polynucleotide, wherein the first probe polynucleotide further comprises a first tag sequence which is bound to a first tag-binding polynucleotide of a first tag-binding conjugate, wherein said first tag-binding conjugate further comprises a first semiconductor nanocrystal conjugated to the first tag-binding polynucleotide.

40. A kit comprising:
a substrate comprising a first target, wherein the first target comprises a polynucleotide;
a first tag-binding conjugate, wherein said first tag-binding conjugate comprises a first semiconductor nanocrystal conjugated to a first tag-binding polynucleotide, and further wherein said first tag-binding conjugate only binds to the first target in the presence of a first probe comprising a first probe polynucleotide comprising a first tag sequence and a first target-binding sequence;
a housing for retaining the substrate and the first tag-binding conjugate;
instructions provided with said housing that describe how to use the components of the kit to assay a sample for the first probe.

41. The kit of claim 40, further comprising a tag polynucleotide comprising a first tag sequence which binds to the tag-binding polynucleotide and does not directly bind to the first target;
wherein the housing further retains the tag polynucleotide; and
wherein the instructions further describe how to use the tag polynucleotide to prepare the probe polynucleotide comprising the first tag sequence from the sample.

42. The kit of claim 41, wherein the substrate is a microarray comprising a plurality of targets, wherein the plurality of targets comprises different targets.

43. The kit of claim 41, wherein the substrate comprises a plurality of different beads, wherein a different target is attached to each of the different beads.

44. The method of claim 1, wherein the target is a polynucleotide located in a cell, which may be fixed or unfixed.


SEQUENCE LISTING (15 sequences)

SEQ ID NO: 1 - DNA, 70 bp, Homo sapiens
ttgagcagtgggctcactctgaagacctgcagtccctcctgcttagggtcgctaatgctgtttcggtgaa

SEQ ID NO: 2 - DNA, 70 bp, Homo sapiens
ccgcgccgacaaacagaacctggaggccattctgcacagcctgcccgagaactgtgccagctggcagtga

SEQ ID NO: 3 - DNA, 70 bp, Homo sapiens
gctcccagaatttcagcttcagcttaactgacagatgttaaagctttctggttagattgttttcacttgg

SEQ ID NO: 4 - DNA, 70 bp, Homo sapiens
ccacctgtccctcctgggctgctggattgtctcgttttcctgccaaataaacaggatcagcgctttaaaa

SEQ ID NO: 5 - DNA, 50 bp, Homo sapiens
ttcaccgaaacagcattagcgaccctaagcaggagggactgcaggtcttc

SEQ ID NO: 6 - DNA, 50 bp, Homo sapiens
tcactgccagctggcacagttctcgggcaggctgtgcagaatggcctcca

SEQ ID NO: 7 - DNA, 50 bp, Homo sapiens
ccaagtgaaaacaatctaaccagaaagctttaacatctgtcagttaagct

SEQ ID NO: 8 - DNA, 50 bp, Homo sapiens
ttttaaagcgctgatcctgtttatttggcaggaaaacgagacaatccagc

SEQ ID NO: 9 - DNA, 69 bp, Artificial Sequence
  Description of Artificial Sequence Complement oligo with generic tag
ggcgtggcggggaaagcatttcaccgaaacagcattagcgaccctaagcaggagggactgcaggtcttc

SEQ ID NO: 10 - DNA, 63 bp, Artificial Sequence
  Description of Artificial Sequence Complement oligo with generic tag
gggcggcgaccttttcaccgaaacagcattagcgaccctaagcaggagggactgcaggtcttc

SEQ ID NO: 11 - DNA, 63 bp, Artificial Sequence
  Description of Artificial Sequence Complement oligo with generic tag
gggcggcgacctttcactgccagctggcacagttctcgggcaggctgtgcagaatggcctcca

SEQ ID NO: 12 - DNA, 69 bp, Artificial Sequence
  Description of Artificial Sequence Complement oligo with generic tag
ggcgtggcggggaaagcattcactgccagctggcacagttctcgggcaggctgtgcagaatggcctcca

SEQ ID NO: 13 - DNA, 29 bp, Artificial Sequence
  Description of Artificial Sequence Oligo for attachment to SCNCs
ctggaacaacactcacaaggtcgccgccc

SEQ ID NO: 14 - DNA, 36 bp, Artificial Sequence
  Description of Artificial Sequence Oligo for attachment to SCNCs
ctggaacaacactcacaatgctttccccgccacgcc

SEQ ID NO: 15 - DNA, 41 bp, Artificial Sequence
  Description of Artificial Sequence Reverse transcription primer including bacteriophage 186 cos site
ggcgtggcggggaaagcatttttttttttttttttttttvn